(19) United States
(12) Patent Application Publication
(10) Pub. No.: US 2025/0367353 A1
(43) Pub. Date: Dec. 4, 2025

(54) PERFUSION FOR TREATMENT OF MEDICAL CONDITIONS

(71) Applicant: Bexorg, Inc., New Haven, CT (US)

(72) Inventor: Zvonimir Vrselja, New Haven, CT (US)

(21) Appl. No.: 19/220,975

(22) Filed: May 28, 2025

Related U.S. Application Data
(60) Provisional application No. 63/655,484, filed on Jun. 3, 2024.

(51) Int. Cl. A61M 1/36; G16H 20/40

(52) U.S. Cl. CPC A61M 1/3613; A61M 1/3663; A61M 1/369; G16H 20/40; A61M 2202/0007; A61M 2202/0208; A61M 2205/3303

(57) ABSTRACT

A perfusion system for treatment of a patient experiencing at least one medical condition includes a reservoir configured to receive perfusate, a cannula configured to fluidly couple the reservoir to an arterial blood vessel of a mammal through a single entry point, a fluid line fluidly coupling the reservoir to the cannula, a pressure sensor configured to measure pressure along the fluid line, a flow sensor configured to measure a flow rate along the fluid line; and a pulse generation system. The pulse generation system includes a pulse generator configured to generate pulsatile flow of perfusate from the reservoir to the cannula along the fluid line based on one or more signals generated by at least one of the pressure sensor or the flow sensor. The perfusion system is configured to perfuse an entire circulatory system of the mammal to treat a patient experiencing at least one medical condition.

### CROSS-REFERENCE TO RELATED APPLICATIONS

This application claims the benefit of priority to U.S. Provisional Application No. 63/655,484, filed on Jun. 3, 2024, the contents of which are hereby incorporated by reference.

### TECHNICAL FIELD

This specification relates to methods, systems, and compositions for treating a patient experiencing a particular medical condition, such as a hemorrhage, with whole body perfusion.

### BACKGROUND

Perfusion in mammalian circulatory systems refers to the process of delivering perfusate (e.g., blood or an artificial perfusate) to the circulatory system of a mammal to supply inputs such as, e.g., oxygen, glucose, and nutrients. The process of perfusion is facilitated by a dense network of blood vessels, including arteries, capillaries, and veins.

### SUMMARY

In one aspect, a perfusion system for treatment of a patient experiencing at least one medical condition includes a reservoir configured to receive perfusate, a cannula configured to fluidly couple the reservoir to an arterial blood vessel of a mammal through a single entry point, a fluid line fluidly coupling the reservoir to the cannula, a pressure sensor configured to measure pressure along the fluid line, a flow sensor configured to measure a flow rate along the fluid line; and a pulse generation system. The pulse generation system includes a pulse generator configured to generate pulsatile flow of perfusate from the reservoir to the cannula along the fluid line based on one or more signals generated by at least one of the pressure sensor or the flow sensor. The perfusion system is configured to perfuse an entire circulatory system of the mammal through the single entry point to treat a patient experiencing at least one medical condition.

Implementations can include one or more of the following features in any combination.

In some implementations, the pulse generation system is controlled based on an internal resistance of the circulatory system of the mammal detected by the pressure sensor.

In certain implementations, the system includes at least one vital sign sensor configured to measure at least one of blood pressure, blood oxygen concentration, heart rate, or core temperature.

In some implementations, the pulse generation system is controlled based on one or more signals generated by the at least one vital sign sensor.

In certain implementations, the system further includes a heat exchanger configured to control a composition of gasses within the perfusate; and the heat exchanger is controlled based at least partly on one more signals generated by the at least one vital sign sensor.

In some implementations, the system further includes a gas mixer configured to control a composition of gasses within the perfusate; and the gas mixer is controlled based at least partly on one more signals generated by the at least one vital sign sensor.

In certain implementations, the mammal is a human.

In some implementations, the system includes a syringe pump manifold fluidly coupled to reservoir, and the syringe pump manifold is configured to introduce one or more substances into the perfusate in the reservoir.

In certain implementations, the system further includes at least one vital sign sensor configured to measure one or more vital signs of the mammal; and the syringe pump manifold is configured to introduce the one or more substances into the perfusate in the reservoir based on one or more signals generated by one the at least one vital sign sensor.

In some implementations, the one or more substances include an oxygen carrier substance.

In certain implementations, the oxygen carrier substance includes erythrocruorin derived from Lumbricus terrestris.

In some implementations, the system further includes a hematocrit sensor fluidly coupled to the fluid line downstream of the pulse generator; and the syringe pump manifold is configured to introduce the one or more substances into the perfusate in the reservoir based on one or more signals generated by the hematocrit sensor.

In certain implementations, the system includes a pump fluidly coupled to the reservoir and to the pulse generator, wherein the pump is configured to flow perfusate from the reservoir to the pulse generator.

In some implementations, the perfusion system is configured to increase the blood pressure of the mammal.

In certain implementations, the perfusate is an acellular composition.

In some implementations, the system includes an air supply system fluidly coupled the pulse generation system, and the air supply system is configured to provide pressurized air to the pulse generator of the pulse generation system based on one or more signals generated by at least one of the pressure sensor or the flow sensor.

In certain implementations, the air supply system includes an air source; and an electronic pressure regulator fluidly coupled to the electronic pressure regulator, the electronic pressure regulator configured to regulate a pressure of a stream of air provided by the air source.

In some implementations, the system includes a pressure sensor coupled to a fluid line downstream of the electronic pressure regulator, and the electronic pressure regulator is controlled based on one or more signals generated by the pressure sensor.

In certain implementations, the medical condition includes a hemorrhage.

In some implementations, the at least one medical condition includes two or more medical conditions.

In certain implementations, the at least one medical condition includes at least one of a heart attack, anemia, an ischemic stroke, peripheral vascular disease, trauma, or respiratory failure.

In another aspect, a method of performing perfusion of a circulatory system of a mammal includes inserting a cannula of a perfusion system into an arterial blood vessel of a mammal experiencing at least one medical condition, controlling a pulse generation system to provide pulsatile flow of perfusate along a fluid line fluidly coupled to the cannula, and adjusting the pulsatile flow of the perfusate based on signals generated by at least one of a pressure sensor coupled to the fluid line or a flow sensor coupled to the fluid line.

Implementations can include one or more of the following features in any combination.

In some implementations, the signals generated by at least one of a pressure sensor coupled to the fluid line or a flow sensor coupled to the fluid line indicate an internal resistance of an arterial system of the mammal.

In certain implementations, the pulse generation system includes a pulse generator and an air supply system fluidly coupled to the pulse generator, and adjusting the pulsatile flow of the perfusate includes controlling the air supply system to supply pressurized air to pulse generator at a particular frequency determined based on signals generated by the at least one of the pressure sensor or the flow sensor.

In some implementations, the method includes controlling a temperature of the perfusate to using a heat exchanger of the perfusion system.

In certain implementations, the method includes controlling a concentration of oxygen in the perfusate using a gas mixer of the perfusion system.

In some implementations, the method includes controlling a syringe pump manifold to add one or more therapeutic compounds to the perfusate.

In certain implementations, the method further includes monitoring one or more vital signs of the mammal, and controlling the syringe pump manifold to add the one or more therapeutic compounds to the perfusate includes controlling the syringe pump manifold based on the one or more vital signs.

In some implementations, the one or more therapeutic compounds include an enhanced oxygen carrier compound.

In certain implementations, the method further includes measuring an oxygen saturation of the mammal; and the syringe pump manifold is controlled based on the measured oxygen saturation of the mammal.

In some implementations, measuring the oxygen saturation of the mammal includes measuring the oxygen saturation using a vital sign sensor coupled to the mammal.

In certain implementations, measuring the oxygen saturation of the mammal includes measuring the oxygen saturation using a hematocrit detector coupled to the fluid line.

In some implementations, adjusting the pulsatile flow of the perfusate based on signals generated by at least one of a pressure sensor coupled to the fluid line or a flow sensor coupled to the fluid line includes monitoring a blood pressure of the mammal based on data generated by the pressure sensor; and adjusting the pulsatile flow of the perfusate based on the blood pressure of the mammal.

In certain implementations, the method further includes monitoring one or more vital signs of the mammal using at least one vital sign sensor; and the pulsatile flow of the perfusate is adjusted further based on data generated by the at least one vital sign sensor.

In some implementations, adjusting the pulsatile flow of the perfusate based on signals generated by at least one of a pressure sensor coupled to the fluid line or a flow sensor coupled to the fluid line includes providing the signals generated by the at least one of the pressure sensor or the flow sensor to a trained machine learning model.

In certain implementations, the method includes training the machine learning model. Training the machine learning model includes inputting a plurality of data generated by at least one vital sign sensor, the pressure sensor, and the flow sensor to the machine learning model as training data; and training the machine learning model, based on the training data, to detect one or more vital signs of the mammal based on data generated by the at least one of the pressure sensor or the flow sensor.

In some implementations, the mammal is a human.

In certain implementations, the mammal is a pig.

In some implementations, the medical condition includes a hemorrhage.

In certain implementations, the at least one medical condition includes two or more medical conditions.

In some implementations, the at least one medical condition includes at least one of a heart attack, anemia, an ischemic stroke, peripheral vascular disease, trauma, or respiratory failure.

Implementations can include one or more of the following advantages. The perfusion systems and methods described herein enable resuscitation of a patient experiencing a hemorrhage through perfusion of a perfusate solution through a single entry point into the patient's circulatory system. For example, perfusion systems and methods described herein can enable the blood volume of a patient experiencing a hemorrhage to be restored and perfusion of the organs of the patient to be maintained. In addition, the perfusion systems and methods described herein enable resuscitation of a patient experiencing a hemorrhage to be performed for 6 hours or longer. As a result, the chances of survival for a patient experiencing a hemorrhage is significantly increased.

The perfusion systems and methods described herein can also be used to administer one or more therapeutic compounds to a patient experiencing a hemorrhage. As a result, the risk of vascular injury resulting from the hemorrhage can be reduced. In addition, perfusion systems and methods described herein can be used to administer oxygen carrier compounds to patient experiencing a hemorrhage. As a result, the oxygen transfer rate of the patient can be maintained and hemodilution can be avoided while the patient is undergoing whole body perfusion to maintain euvolemic control of the patient's circulatory system.

The perfusion systems and methods described herein can also be used to provide hemodynamic, vascular, and metabolic support to a patient experiencing non-hemorrhage injuries or conditions. As a result, the chance of survival of a patient experiencing non-hemorrhage injuries or conditions and the risk of vascular injury to the patient resulting from the non-hemorrhage injuries or conditions is reduced.

The perfusion systems and methods described herein can also help prevent cellular damage induced by ischemic conditions caused by or related to one or more medical conditions, such as a hemorrhage, a heart attack, or stroke-induced ischemia

The details of certain implementations are set forth in the accompanying drawings and the description below. Other aspects, features, and advantages will be apparent from the description and drawings, and from the claims.

### BRIEF DESCRIPTION OF DRAWINGS

FIG. 1 is a schematic illustration of a perfusion system for treating a mammal during an ongoing hemorrhage.

FIG. 2A is a perspective view of an example pulse generator of the system of FIG. 1.

FIG. 2B is an exploded view of the example pulse generator of FIG. 2A.

FIG. 2C is a side view of the example pulse generator of FIG. 2A.

FIG. 2D is a cross-sectional view of the pulse generator of FIG. 2A along axis A-A depicted in FIG. 2C.

FIG. 3 depicts example blood flow and blood pressure measurements recorded during an example perfusion treatment using the system of FIG. 1.

FIG. 4 depicts example blood flow measurements, example blood pressure measurements, intervention pressure measurements, and intervention flow measurements recorded during an example perfusion treatment using the system of FIG. 1.

FIG. 5 is a block diagram of an example computer system by which a computer system of the perfusion system of FIG. 1 can be implemented.

Like references symbols in the various drawings indicate like elements.

### DETAILED DESCRIPTION

This specification generally describes devices, systems, and methods for perfusion of circulatory systems. In particular, this specification describes devices, systems, and methods for perfusing a human circulatory system to treat a patient experiencing a hemorrhage, for example as a result of vascular trauma.

An example of an electromechanical perfusion device is described in: Zvonimir Vrselja et al., “Restoration of brain circulation and cellular functions hours postmortem,” Nature, 2019 April; 568 (7752), which is incorporated by reference herein. Examples of electromechanical perfusion devices and artificial perfusion experiments are also described in U.S. patents application Ser. Nos. 16/967,925 and 63/562,094, which are incorporated by reference herein.

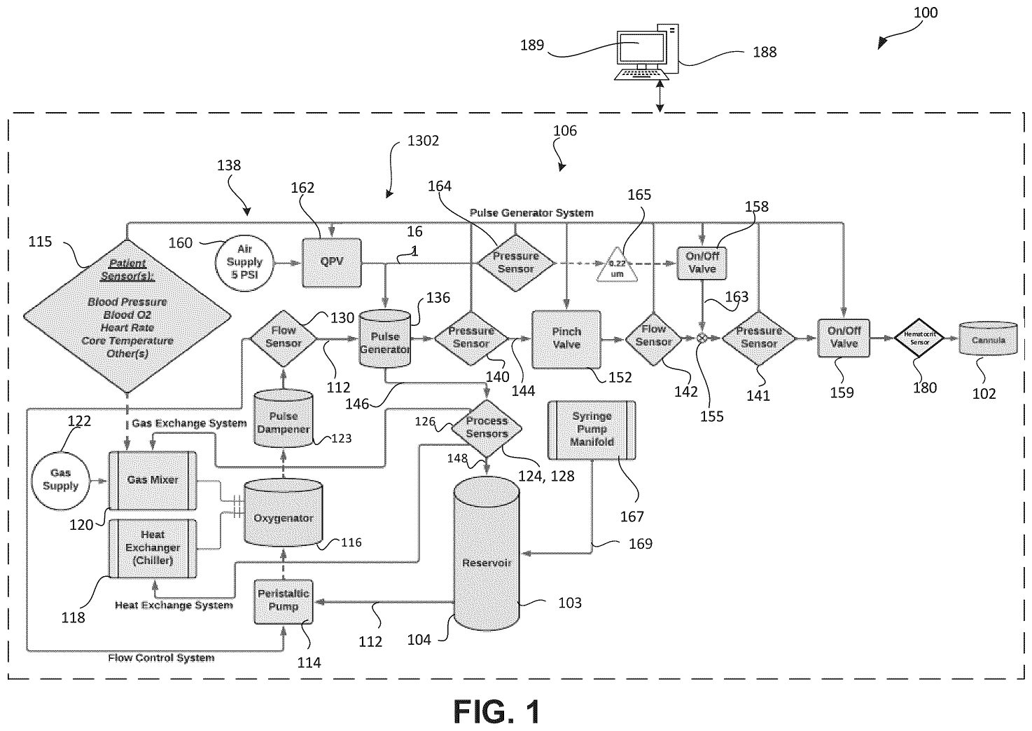

FIG. 1 depicts an example perfusion system 100 for perfusion of a circulatory system of a patient that is experiencing a hemorrhage in order to resuscitate and sustain the patient until the hemorrhage is treated. The perfusion system 100 includes a perfusate reservoir 104 that is configured to contain a perfusate solution (also referred to herein as “perfusate”) and a cannula 102 that is configured to fluidly couple the perfusate reservoir 104 to the circulatory system of a patient. As will be described in further detail herein, the perfusate contained within the perfusate reservoir 104 is an acellular, whole blood substitute that is configured to perfuse the entire circulatory system of the patient in order to help sustain a patient experiencing a hemorrhage by maintaining the patient's blood pressure and blood flow rate within normal ranges.

The system 100 also includes a plurality of patient vital sign sensors 115 configured to measure one or more physiological vital signs of a patient being treated with the system 100 throughout the perfusion process. The vital sign sensors 115 are configured to measure patient physiological characteristics including, but not limited to, blood pressure, blood oxygen concentration (also referred to herein as O₂ partial pressure), heart rate, and core body temperature. In some implementations, the vital sign sensors 115 include an oxygen monitor, a heartbeat monitor, a blood pressure monitor, and a core temperature monitor. As will be described in further detail herein, a heat exchanger 118, gas mixer 120, and pulse generation system 1302 of the system 100 are controlled based on the patient vital signs measured by the vital sign sensors 115.

The cannula 102 is fluidly connected to the perfusate reservoir 104 through an arterial circuit 106 that is configured to circulate a synthetic acellular perfusate under a pulsatile flow. The arterial circuit 106 includes an arterial fluid line 112 and an perfusion line 144 fluidly coupling the perfusate reservoir 104 to the cannula 102, and the fresh perfusate contained within the perfusate reservoir 104 is provided to the cannula 102 along the arterial fluid line 112 and the perfusion line 144. As will be described in greater detailed herein, through insertion of the cannula 102 into an arterial blood vessel of a patient, the cannula 102 enables perfusion of the patient's entire circulatory system (also referred to herein as “whole body perfusion”) through a single point of entry into the patient's circulatory system. In some implementations, the cannula 102 is configured to be inserted into the femoral artery or the brachial artery of the patient.

Referring to FIG. 1, the arterial circuit 106 also includes a flow pump 114 positioned along the arterial fluid line 112 between the perfusate reservoir 104 and an oxygenator 116. The flow pump 114 is a peristaltic pump configured to pump fresh perfusate from the perfusate reservoir 104 to the oxygenator 116. The peristaltic flow pump 114 is configured to minimize shear of the perfusate as it is pumped along the arterial fluid line 112.

The oxygenator 116 includes a heat exchanger 118 and a gas mixer 120. The heat exchanger 118 includes one or more metal heat exchanging coils configured to regulate the temperature of the perfusate received by the oxygenator 116. As will be described in further detail herein, the arterial circuit 106 includes a sensor block 124 with sensor probes 128 configured to measure certain characteristics of the fresh perfusate flowing through the arterial circuit 106, including the temperature of the fresh perfusate, and the heat exchanger 118 is configured to adjust the temperature of the perfusate flowing through the heat exchanger 118 based on the temperature of the perfusate detected by the sensor block 124. For example, the heat exchanger 118 is configured to adjust the temperature of the perfusate to be within a predetermined range of temperature based on the temperature signals generated by the sensor block 124. In some implementations, the heat exchanger 118 is configured to heat the perfusate to a temperature corresponding to a normal physiological body temperature. For example, the heat exchanger 118 can be configured control the temperature of the perfusate to be within a range of 16° C. to 37° C.

The system 100 includes a plurality of patient vital sign sensors 115 in communication with the heat exchanger 118, and the heat exchanger 118 can be controlled based on one or more signals generated by one or more of the vital sign sensors 115. For example, the vital sign sensors 115 can include a temperature sensor configured to measure the core body temperature of the patient, and the heat exchanger 118 can be controlled to increase or decrease the temperature of the perfusate based on the core body temperature of the patient measured by the vital sign sensors 115.

The gas mixer 120 of the oxygenator 116 is fluidly coupled to and receives one or more gasses from a gas source 122, and the gas mixer 120 is configured to dissolve the one or more gasses into the perfusate flowing through the oxygenator 116. The gas mixer 120 includes three inlets fluidly coupled to the gas source 122. A first inlet of the gas mixer 120 is configured to be fluidly coupled to a source of O₂, a second inlet of the gas mixer 120 is configured to be coupled to a source of N₂, and a third inlet of the gas mixer 120 is configured to be coupled to a source of CO₂. The gas mixer 120 is controlled based on one or more signals generated by the sensor block 124 indicating the dissolved gas levels of the perfusate within the sensor block 124. For example, the sensor block 124 can generate one or more signals indicating the dissolved oxygen concentration of the perfusate within the sensor block 124, the dissolved nitrogen concentration of the perfusate within the sensor block 124, and the dissolved carbon dioxide concentration of the perfusate within the sensor block 124 and, based on the signals, the gas mixer 120 adjusts the amount of oxygen, nitrogen, and/or carbon dioxide provided to the perfusate within the oxygenator 116 in order to control the dissolved oxygen concentration, the dissolved nitrogen concentration, and the dissolved carbon dioxide concentration of the perfusate to be within a predetermined range. For example, based on the signals generated by the sensor block 124, the gas mixer 120 generates commands to control internal gas valves of the gas mixer 120 to control the flow rate of gas into each respective inlet of the gas mixer 120 in order to control the amount and rate that each gas (O₂, N₂, and CO₂) are provided to the oxygenator 116.

In addition, the gas mixer 120 monitors the pressure and flow rate of gas provided from the gas mixer 120 to the oxygenator 116. In some implementations, the flow rate of gas from the gas mixer 120 to the oxygenator 116 and the flow rate of perfusate to the oxygenator 116 are each controlled to maintain a predetermined ventilation/perfusion coefficient. For example, in some implementations, the gas mixer 120 maintains the flow rate of gas from the gas mixer 120 to the oxygenator 116 at approximately 700 mL/minute. As described herein, in some implementations, the flow pump 114 is configured to flow the perfusate to the oxygenator 116 at a rate of 400 mL/minute. By flowing the gas into the oxygenator 116 at a rate of 700 mL/minute and simultaneously flowing perfusate into the oxygenator 116 at a rate of 400 mL/minute, a predetermined ventilation/perfusion coefficient of 700/400 can be maintained throughout the perfusion process. By adjusting the flow rate of each of the gasses flowing into the gas mixer 120 while maintaining the rate of gas flowing out of the gas mixer 120 into oxygenator 116, the₂ gas mixer 120 can control and adjust the concentration of each of the respective gasses within the perfusate while maintaining the predetermined ventilation/perfusion coefficient.

The concentration of the CO₂, N₂, and O₂ within the perfusate is maintained within physiological limits, as determined based on the concentration detected using the sensor probes 128 in the sensor block 124. In some implementations, the gas mixer 120 and oxygenator 116 are controlled based on an alpha stat approach to maintain the blood gas concentrations within normal, physiological limits. In some implementations, the partial pressure of CO₂ within the partial pressure of the CO₂ within the perfusate is maintained at a partial pressure of approximately 400 mmHg during the perfusion process. In some implementations, the partial pressure of O₂ in the perfusate is maintained at approximately 150 to 200 mmHg during the perfusion process.

The vital sign sensors 115 are communicably coupled to the gas mixer 120, and the gas mixer 120 is configured to adjust the concentration of one or more gasses within the perfusate based on signals received from the vital sign sensors 115. For example, the vital sign sensors 115 can include one or more sensors configured to measure the partial pressure of oxygen within the patient's blood (i.e., oxygen saturation) and the gas mixer 120 is controlled to adjust the partial pressure of O₂ within the perfusate based on the measured oxygen saturation of the patient. For example, the gas mixer 120 is configured to adjust the partial pressure of O₂ within the perfusate dynamically in order to maintain the patient's oxygen saturation to normal physiological levels during perfusion of the patient. While a patient is experiencing a hemorrhage, the oxygen saturation of the patient's tissue typically decreases below normal physiological levels as a result of blood loss. In order to restore the patient's oxygen saturation during an active hemorrhage, the gas mixer 120 can be controlled to increase the partial pressure of O₂ of the perfusate provided to the patient until the vital sign sensors 115 detect that the patient's oxygen saturation is within normal physiological limits.

The arterial circuit 106 includes a pulse dampener 123 fluidly coupled to the arterial fluid line 112 downstream of the oxygenator 116. Fluid flowing from the oxygenator 116 passes through the pulse dampener 123 and the pulse dampener 123 reduces the pulsations and high frequency oscillations in the flow of perfusate along the fluid line 112 that are generated by the peristaltic pump 114. By flowing the perfusate exiting the oxygenator 116 through the pulse generator, any fluidic turbulence in the flow of perfusate generated by the oxygenator 116 is reduced and a laminar flow of perfusate toward the pulse generator 136 is promoted.

The arterial circuit 106 includes a flow sensor 130 positioned along the arterial fluid line 112 downstream of the oxygenator 116 and the pulse dampener 123. When the flow pump 114 is operated, fresh perfusate exiting the pulse dampener 123 flows along the arterial fluid line 112 through the flow sensor 130, and the flow pump 114 is controlled based on signals generated by the flow sensor 130. In some implementations, the pump speed of the flow pump 114 is adjusted based on one or more signals received from the flow sensor 130 indicating the flow rate of the perfusate exiting the pulse dampener 123 in order to control the flow rate of the perfusate exiting the pulse dampener 123 to be within a predetermined range. In some implementations, the flow pump 114 is controlled to maintain the flow rate of perfusate exiting the pulse dampener 123 to be 400 mL/minute. The flow rate of perfusate into the oxygenator 116 and the flow rate of gas into the oxygenator 116 can each be controlled to maintain a predetermined ventilation/perfusion coefficient. Thus, in some implementations, the flow pump 114 and the gas mixer 120 are each controlled to adjust the flow rates of perfusate and gas, respectively, into the oxygenator 116 in order to maintain the predetermined ventilation/perfusion coefficient.

The arterial circuit 106 also includes a pulse generation system 1302 configured to convert laminar flow of perfusate along the arterial fluid line 112 to pulsatile flow along the perfusion line 144. Referring to FIG. 1, pulse generation system 1302 includes a pulse generator device 136 (also referred to herein as “pulse generator 136”) and an air supply system 138 fluidly coupled to the pulse generator 136. After flowing out of the pulse dampener 123, the perfusate flows along the arterial fluid line 112 to the pulse generator 136. The pulse generator 136 is configured to convert the steady state flow of the fresh perfusate along the arterial fluid line 112 to pulsatile flow. As a result, fresh perfusate is provided through the cannula 102 into the patient's arterial system as a pulsatile flow that more closely mimics the anatomical flow of blood within the human arterial system, which results in improved perfusion of the patient's circulatory system. As will be described in detail herein, the pulsatile flow of perfusate generated by the pulse generation system 1302 is dynamically adjusted throughout the perfusion process to optimize the flow rate and fluid pressure of perfusate provided to the patient's circulatory system responsive to the level of resistance of the patient's arterial system, which improves the perfusion of the patient's circulatory system and reduces the risk of vascular injury during perfusion.

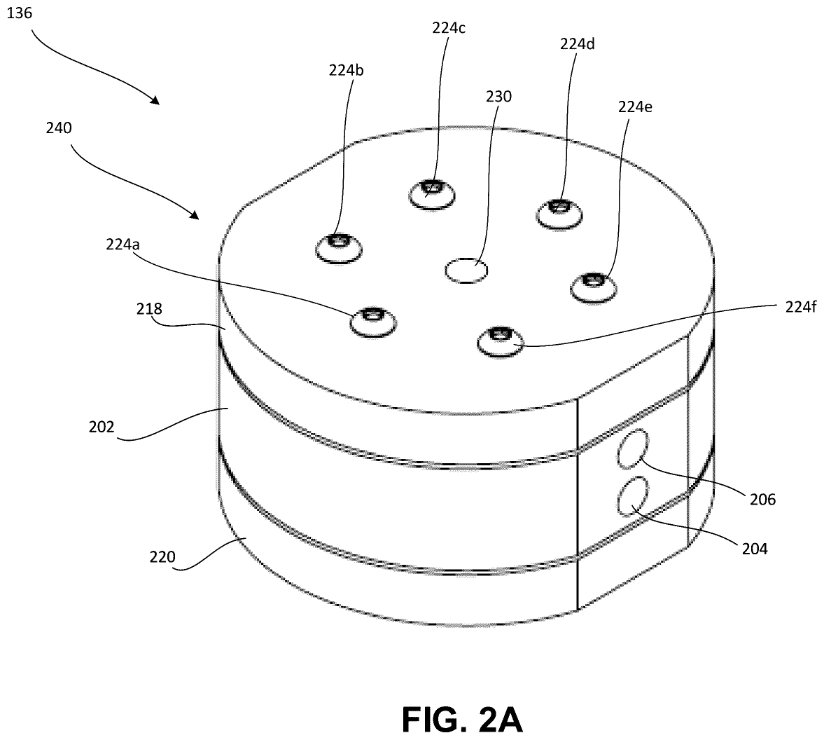

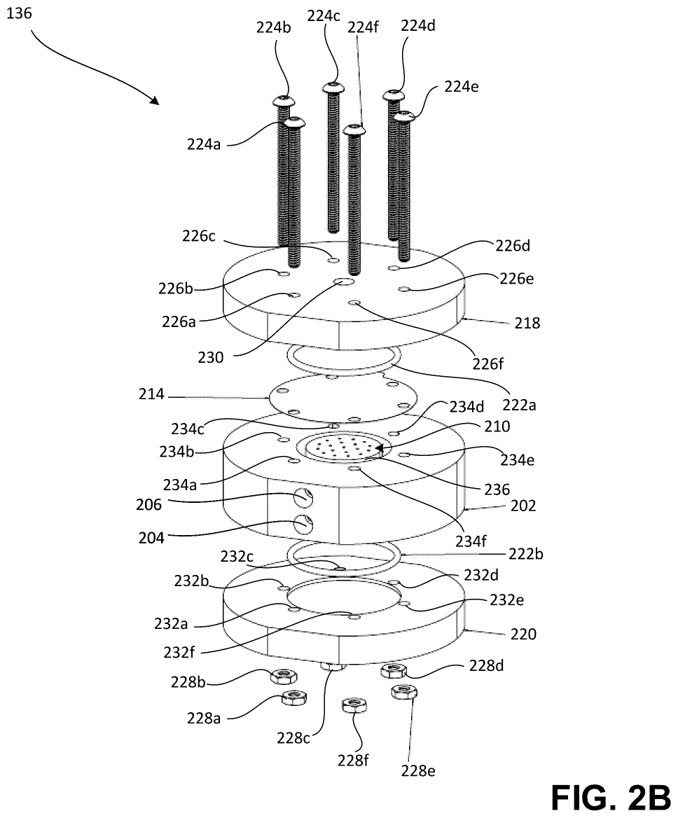

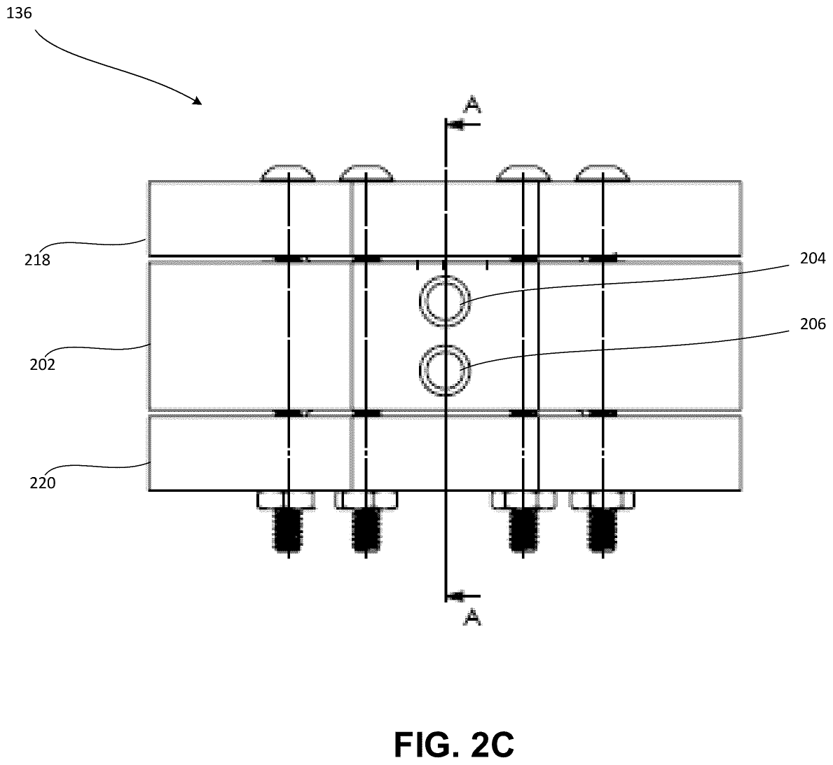

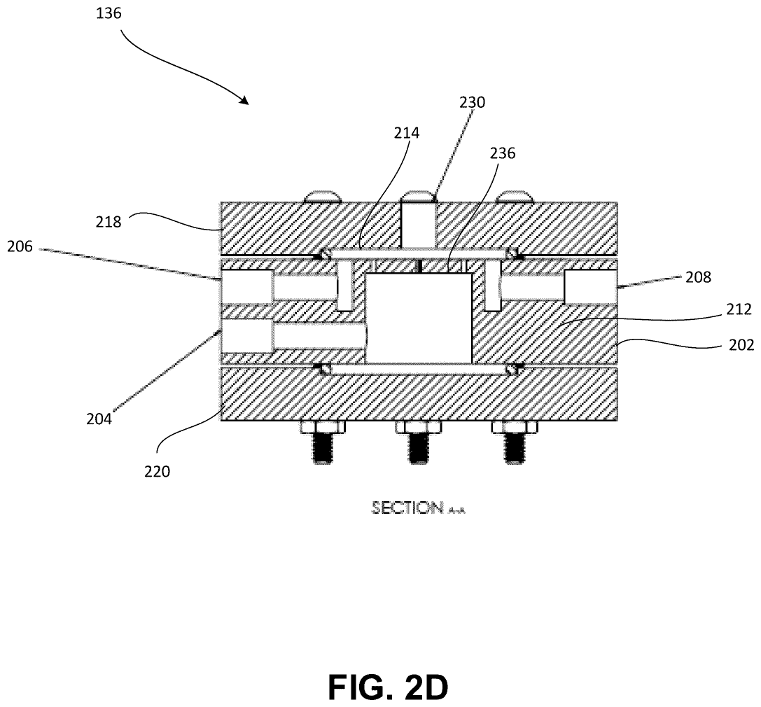

FIGS. 2A-2D depict the example pulse generator device 136. The pulse generator 136 includes a body 240 that defines a perfusate inlet 204, a sensor block outlet 206, and an perfusion line outlet 208. Referring to FIGS. 1 and 2A-2D, the perfusate inlet 204 is fluidly coupled to a fluid chamber 236 of the pulse generator 136. The fluid chamber 236 is configured to receive perfusate flowing into the pulse generator 136 from the arterial fluid line 112 through the perfusate inlet 204. The fluid chamber 236 includes a plurality of openings 210 through an upper surface of the fluid chamber 236, and perfusate received by the fluid chamber 236 from the perfusate inlet 204 is configured to flow through the openings 210 into the interior 212 of a central housing 202 of the pulse generator 136. Fluid contained within the interior 212 of the central housing 202 flows through the sensor block outlet 206 and/or the perfusion line outlet 208, as will be described herein. In some implementations, the diaphragm 214 is a back pressure regulator membrane manufactured by EQUILIBAR®.

Referring to FIG. 2B, the pulse generator 136 further includes a lid 218 and a base 220. A flexible diaphragm 214 is positioned over the fluid chamber 236 between the lid 218 and the central housing 202 of the pulse generator 136. The lid 218 is coupled to an upper surface of the central housing 202 and the base 220 is coupled to a lower surface of the central housing 202. O-rings 222a, 222b are positioned between the lid 218 and the central housing 202 and between the base 220 and the central housing 202, respectively, in order to prevent perfusate contained within the interior 212 of the central housing 202 from leaking outside of the central housing 202. The lid 218 and the base 220 are coupled to the central housing 202 by a plurality of bolts 224a, 224b, 224c, 224d, 224e, 224f (collectively referred to herein as bolts 224) inserted through a series of respective openings 226a, 226b, 226c, 226d, 226e, 226f through the lid 218, respective openings 234a, 234b, 234c, 234d, 234e, 234f through the central housing 202, and respective openings 232a, 232b, 232c, 232d, 232e, 232f through the base 220. A respective plurality of nuts 228a, 228b, 228c, 228d, 228e, 228f are to the bolts 224.

The lid 28 of the pulse generator 136 defines an air inlet 230 fluidly coupled to and configured to receive pressurized air from the air supply system 138. The air supply system 138 is configured to provided pressurized air to the pulse generator 136 in order to control the pulsatile flow of perfusate received by pulse generator 136. For example, the diaphragm 214 flexes downwards towards the fluid chamber 236 in response to pressurized air being provided by the air supply system 138 through the air inlet 230 of the pulse generator 136. The flexure of the diaphragm 214 causes the openings 210 in the fluid chamber 236 to be covered by the diaphragm 214 and increases the pressure within the interior 212 of the central housing 202, which results in increased flow through the perfusion line outlet 208 of the pulse generator 136 to the perfusion line 144 that fluidly couples the pulse generator to the cannula 102.

Referring to FIG. 1, the air supply system 138 includes an air source 160, an electronic pressure regulator 162, and a pressure sensor 164 positioned downstream of the electronic pressure regulator 162. The air source 160, the electronic pressure regulator 162, and the pulse generator 136 are each fluidly coupled along an air supply line 161.

The electronic pressure regulator 162 is controlled based on pressure signals received from the pressure sensor 164 in order to provide a pressurized stream of air in a particular pressure range to the air inlet 230 of the pulse generator 136. The pressure of the air stream generated by the electronic pressure regulator 162 is controlled based on pressure signals received from the pressure sensor 164 of the air supply system 138. In addition, as will be described in further detail herein, the electronic pressure regulator 162 is controlled based on signals received from a pressure sensor 141 and a flow sensor 142 positioned along the perfusion line 144 that fluidly couples the pulse generator 136 to the patient's circulatory system. In some implementations, the electronic pressure regulator 162 is a QPV electronic pressure regulator manufactured by EQUILIBAR®. Prior to conducting a perfusion treatment, the pressure sensor 162 can be calibrated using a manometer fluidly coupled to the arterial circuit 106.

The air supply system 138 also includes an air calibration line 163 that fluidly couples the air supply line 161 to the perfusion line 144 downstream of the pressure sensors 140, 141 positioned along the perfusion line 144. The pressure sensors 140, 141 along the perfusion line 144 are calibrated by opening the solenoid valve 158 and providing pressurized air to the perfusion line 144 along the air calibration line 163. For example, during initial calibration of the pressure sensors 140, 141, the valve 158 along the air calibration line 163 is opened and pressurized air is provided to the perfusion line along the air calibration line 163 through stopcock 155. The valves 152, 159 are both closed to generate a stable air pressure along the perfusion line 144 between the valves 152, 159, and the pressure sensor 141 can be calibrated based on the measured pressure. In some implementations, the pressure sensors 140, 141 are calibrated using a manometer fluidly coupled to the arterial circuit. A filter 165 is positioned along the air calibration line 163 in order to filter out and prevent any particulates in the pressurized air stream flowing along the air calibration line 163 from entering the perfusion line 144. In some implementations, the filter 165 is a 0.22 μm filter.

In order to perform whole body perfusion of a patient, the pulse generator 136 and the air supply system 138 are controlled to provide a pulsatile flow of fresh perfusate through the cannula 102 into the patient's circulatory system within predetermined boundaries of pressure and flow rate that are selected to effectively perfuse the circulatory system of the patient while minimizing damage to the vasculature and other tissues. Referring to FIGS. 1 and 2D, a sensor block inlet line 146 is coupled to the sensor block outlet 206 of the pulse generator 136 to fluidly couple the pulse generator 136 to the sensor block 124. The perfusion line 144 is coupled to the perfusion line outlet 208 of the pulse generator 136 to fluidly couple the pulse generator 136 to the cannula 102, which is fluidly coupled to the patient's circulatory system through insertion of the cannula 102 into an arterial blood vessel of the patient. The sensor block inlet line 146 has a larger diameter than the perfusion line 144. In addition, the perfusion line 144, the cannula 102 and the pulse generator 136 are each coupled to a respective platform that maintains the cannula 102, the perfusion line 144, and the pulse generator 136 at substantially equal heights above the height of the sensor block inlet line 146.

The sensor block inlet line 146 extends downwards relative to the perfusion line 144. As a result of the relative positioning of the pulse generator 136, the cannula 102, the perfusion line 144, and the sensor block 124, there is no passive flow of fluid from the pulse generator 136 to the cannula 102, and all of the perfusate contained within the interior 212 of the central housing 202 of the pulse generator 136 passively flows through the sensor block inlet line 146 when the diaphragm 214 of the pulse generator 136 is in an unflexed (“neutral”) position. Therefore, in order to provide fresh perfusate from the pulse generator 136 to the cannula 102 via the perfusion line 144, the diaphragm 214 of the pulse generator 136 must be flexed downwards towards the fluid chamber 236 to cover the openings 210 in the fluid chamber 236 and increase the pressure within the interior 212 of the central housing 202. As result of the increased pressure caused by flexing the diaphragm 214, at least a portion of the fresh perfusate contained within the interior 212 of the pulse generator is forced through the perfusion line outlet 208 and along the perfusion line 144 to the cannula 102.

In addition, in some implementations, the sensor block inlet line 146 has a larger diameter than the perfusion line 144. In some implementations, the sensor block inlet line 146 includes a Y-connector and two fluid lines extending from the Y-connector to fluidly couple the pulse generator 136 to the sensor block 124, further decreasing the resistance to flow between the pulse generator 136 and the sensor block 124. As a result of the decreased resistance to flow between the pulse generator 136 and the sensor block 124 compared to the resistance to flow between the pulse generator 136 and the cannula 102 due to the structural differences in the sensor block inlet line 146 and the perfusion line 144, at least a portion of the perfusate flowing through the pulse generator 136 is provided to the sensor block 124 from the pulse generator 136, even when the diaphragm 214 of the pulse generator 136 is in a flexed position.

In order to maintain an optimal pulsatile flow of perfusate along the perfusion line that is responsive to the internal resistance of the patient's circulatory system (e.g., the internal resistance of the patient's aorta or arterial system), the air supply system 138 coupled to the pulse generator 136 is controlled based on signals generated by a pressure sensor 141 and a flow sensor 142 positioned along the perfusion line 144. In some implementation, the pulse generation system 1302 is controlled to maintain the pressure along the perfusion line 144, as measured by the pressure sensor 141, between 5 mmHg and 100 mmHg. In some implementation, the pulse generation system 1302 is controlled to maintain the flow rate along the perfusion line 144, as measured by the flow sensor 142, between 0 mL/min and 750 mL/minute. The flow rate measured by the flow sensor 142 corresponds to the average flow rate of the pulse of fluid provided by the pulse generator 136.

The electronic pressure regulator 162 of the air supply system 138 is controlled to provide pressurized air to the air inlet 230 of the pulse generator 136 at particular intervals and durations based on the signals generated the second pressure sensor 141 and the flow sensor 142 along the perfusion line 144 in order to achieve a particular pressure and/or flow rate of perfusate along the perfusion line 144. As the electronic pressure regulator 162 provides pressurized air to the air inlet 230 of the pulse generator 136, the diaphragm 214 of the pulse generator 136 flexes downwards towards the fluid chamber 236 in response to the increased positive pressure in the air inlet 230 and covers the openings 210 in the fluid chamber 236. As result of the increased pressure within the interior 212 of the pulse generator 136 caused by the flexing of the diaphragm 214, at least a portion of the fresh perfusate contained within the interior 212 of the pulse generator is forced through the perfusion line outlet 208 and along the perfusion line 144 to the cannula 102. By adjusting the frequency and/or duration of the pressurized air streams provided by the electronic pressure regulator 162 to the pulse generator 136 in response to pressure and flow rate signals received from sensors 141, 142, the amplitude and base of the pulse of perfusate provided by the pulse generator 136 to the perfusion line 144 can be optimized based on the internal resistance of the patient's circulatory system as detected by the sensor 141, 142. The frequency and/or duration of the pressurized air streams provided by the electronic pressure regulator 162 to the pulse generator 136 are adjusted in real-time throughout perfusion process as the resistance of the patient's vascular system, including the resistance of the patient's aorta, changes throughout the perfusion process, as measured by the pressure sensor 141 and flow sensor 142.

In addition, as depicted in FIG. 1, the perfusion system 100 includes a resistance valve 152 positioned along the perfusion line 144 and that can be operated to further provide a particular flow resistance along the perfusion line 144. In particular, the resistance valve 152 is controlled to prevent passive flow of perfusate along the perfusion line 144 due to the driven flow provided by the flow pump 114.

The resistance valve 152 is positioned along the perfusion line 144 downstream of the first pressure sensor 140 and the flow sensor 142 and is positioned upstream of the second pressure sensor 141. During calibration of the system 100, the flow pump 114 is operated to flow perfusate along the perfusion line 144 toward the cannula 102 at a predetermined rate without operating the pulse generation system 1302. As the flow pump flows perfusate along the perfusion line 144 during calibration, the pressure along the perfusion line 144 upstream of the resistance valve 152 is measured by the first pressure sensor 140 and the pressure along the perfusion line 144 downstream of the resistance valve 152 is measured by the second pressure sensor 141. Based on the pressure measured by the pressure sensors 140, 141, the resistance valve 152 can be adjusted (e.g., partially or fully opened or partially or fully closed) in order to predetermined internal resistance along the perfusion line 144. In some implementations, the resistance valve 152 is controlled, based on the data generated by the pressure sensors 140, 141, to provide an internal resistance of 1 mmHg along the perfusion line 144.

The pressure measured by the first pressure sensor 140 can be used to determine the effectiveness of the pulse dampener 123 at smoothing the flow waveform. In addition, the pressure measured by the first pressure sensor 140 can be used to determine the efficiency of the pulse generator 136 at translating the air pressure waves generated by the air supply system 138 into fluidic waveforms along the perfusion line 144.

During a perfusion treatment, the pulse generation system 1302 is controlled based on data generated by the vital sign sensors 115, the pressure sensor 141, and the flow sensor 142. For example, when a hemorrhage occurs, for example due to traumatic vascular injury, blood escapes the patient's circulatory system and the blood pressure of the patient continually decreases until the hemorrhage is treated. In order to resuscitate and sustain a patient experiencing a hemorrhage, the system 100 is used to perfuse the circulatory system of the patient using a pulsatile flow of perfusate, which increases the blood pressure and blood volume of the patient. In order to control the blood pressure of the patient within a normal physiological range, the pulse generation system 1304 is automatically controlled to increase or decrease the pressure along the perfusion line 144, as measured by the pressure sensor 141, based on the blood pressure of the patient detected by one or more of the vital sign sensors 115. For example, the vital sign sensors 115 continuously measure the patient's blood pressure in real time throughout the perfusion treatment. Based on the blood pressure of the patient measured by the vital sign sensors 115, the pulse generator 136 is controlled to adjust the pressure along the perfusion line 144, as detected by the pressure sensor 141, until the blood pressure of the patient detected by the vital sign sensors 115 is within a normal physiological range.

As the pressure along the perfusion line 144 is measured by the pressure sensor 141, the flow rate of perfusate along the perfusion line 144 is simultaneously monitored using the flow sensor 142, and the pulse generation system 1302 is controlled to adjust the flow rate of perfusate along the perfusion line 144 and through the cannula 102. In order to control the blood pressure of the patient within a normal physiological range during a hemorrhage, the pulse generation system 1302 is automatically controlled to increase or decrease the flow rate of perfusate along the perfusion line 144, as measured by the flow sensor 142, based on the blood pressure and arterial blood flow of the patient measured by one or more of the vital sign sensors 115. For example, the vital sign sensors 115 continuously measure the patient's blood pressure and arterial blood flow rate in real time throughout the perfusion treatment. Based on the blood pressure and arterial blood flow rate of the patient measured by the vital sign sensors 115, the pulse generation system 1302 can be dynamically controlled to adjust the flow rate of perfusate along the perfusion line 144, as detected by the flow sensor 142, until the blood pressure and arterial blood flow of the patient detected by the vital sign sensors 115 are within a normal physiological range.

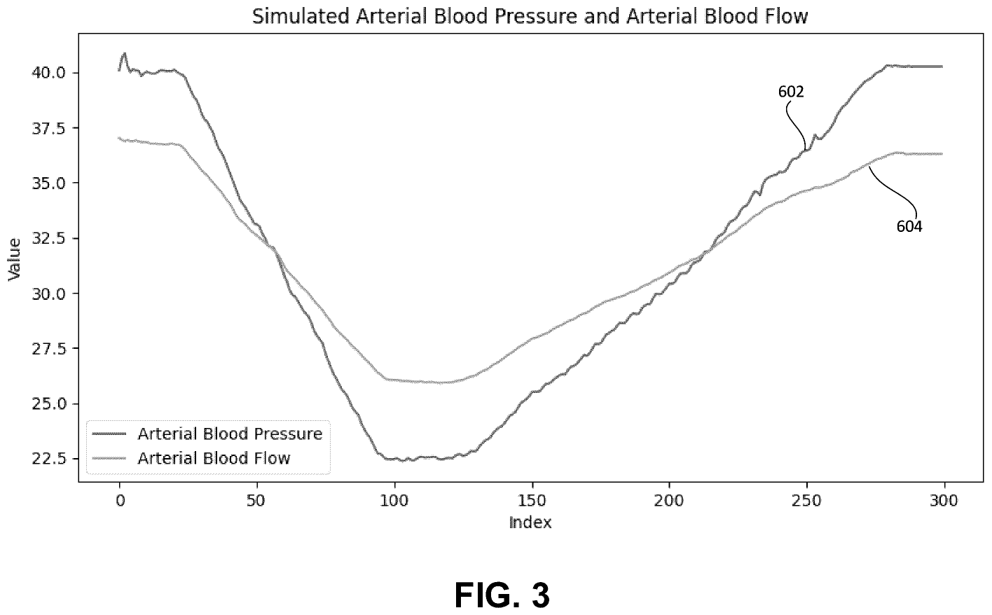

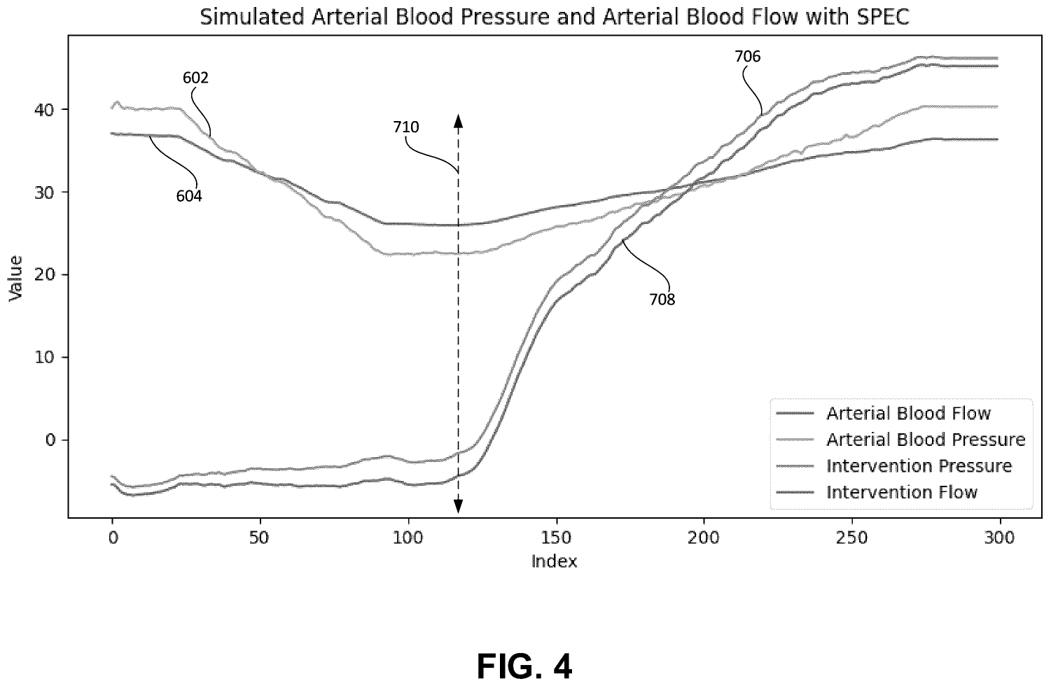

FIGS. 3 and 4 depicts example vital sign sensor data representing the arterial blood pressure 602 and arterial blood flow rate 604 of a pig that was tested with a simulated hemorrhage and treated with circulatory system perfusion using the perfusion system 100 depicted in FIG. 1. During testing, blood was removed from the pig at a predetermined rate in order to simulate a hemorrhage, which resulted in a corresponding decrease in arterial blood pressure 602 and arterial blood flow 604, as depicted in FIG. 3. After a predetermined amount of blood was removed from the pig, the cannula 102 of the perfusion system 100 was inserted into an arterial blood vessel of the pig, and perfusion of the circulatory system of the pig was conducted using the perfusion system 100, as described herein. As can be seen in FIG. 3, perfusion of the circulatory system of the pig using the perfusion system 100 resulted in the arterial blood pressure 602 and arterial blood flow 604 of the pig to recover to normal levels. As demonstrated by this experiment, the perfusion system 100 can provide euvolemic control of a patient by automatically restoring blood flow and blood volume in response to detecting a decrease in arterial resistance and/or a decrease in blood pressure, for example due to the patient experiencing a hemorrhage.

FIG. 4 also includes data depicting the pressure 706 measured along the perfusion line 144 by pressure sensor 141 and the flow rate 708 measured along the perfusion line 144 throughout by the flow sensor 142. At the start 710 of perfusion treatment of the subject experiencing a hemorrhage, the pressure 706 and flow rate 708 along the perfusion line 144 were both increased sharply based on the low blood pressure 602 and arterial blood flow 604 measured by the vital sign sensors 115. As perfusion treatment progressed, the blood pressure 602 and arterial blood flow 604 of the subject, as measured by the vital sign sensors 115, began to recover. Based on the blood pressure 602 and arterial blood flow 604 data generated by the vital sign sensors 115, the pulse generation system 1302 was dynamically controlled to reduce the rate of increase in the pressure 706 and flow rate 708 along the perfusion line 144, as depicted in FIG. 4.

During a perfusion treatment, the pulse generation system 1302 is also controlled throughout the perfusion process based on the signals generated by the pressure sensor 141 and the flow sensor 142 in order to maintain the flow rate and pressure along the perfusion line 144 within predetermined boundaries. For example, while perfusate is provided to the cannula 102 in pulses generated by the pulse generation system 1302, the pressure along the perfusion line 144 is measured by the pressure sensor 141. If the pressure detected by the second pressure sensor 141 falls outside of predetermined pressure boundaries, the pulse generation system 1302 is controlled to continuously adjust the pulsatile flow along the perfusion line 144 until the pressure measured by the second pressure sensor 141 is within the pressure boundaries. In some implementations, the flow rate and pressure boundaries for the perfusion line are defined to minimize fluidic sheer flow and fluidic turbulence of perfusate flowing along the perfusion line 144 and through the cannula 102. In some implementations, the boundaries for the flow rate for the perfusion line are selected based on computational fluid dynamics to ensure that the flow of perfusate along the perfusion line 144 and through the cannula 102 has a Reynolds number within a predetermined range that corresponds to optimal flow dynamics. Because the pulsatile flow provided by the pulse generator 136 is controlled in real-time based on the pressure and the flow rate detected by the second pressure sensor 141 and the flow sensor 142, respectively, the flow of perfusate to patient's circulatory system through the cannula 102 is controlled dynamically in response to the real-time changes in internal resistance of the patient's circulatory system throughout the perfusion process.

As used herein, a real-time operation may describe an operation that is performed with minimal delay, taking into account the limitations of the computing system(s) performing the operation. For example, in some implementations, the flow sensor 142 is sampled at a rate of 100 Hz, the pressure sensors 140, 141 are sampled at a rate of 80 Hz, and the pulse generation system 1302 is controlled adjust the flow of perfusate to the cannula 102 every one second based on the data received from the flow sensor 142 and the pressure sensor 141. In some implementations, the flow sensor 130 is sampled at a rate of 100 Hz and the flow pump 114 is controlled every 0.5 seconds based on the data generated by the flow sensor 130. In some implementations, the sensor probes 128 in the sensor block 124 are each sampled at a rate of 1 Hz, and the operations of the heat exchanger 118 and the gas mixer 120 are each updated every one second based on the data generated by the sensor probes 128. In some implementations, the resistance valve 152 is controlled at a 10 Hz frequency. The data frequency and resolution of the sensors of the perfusion system 100 can be assessed using Fourier analysis and step-wise tests. The dynamic nature of the perfusate flow provided to the cannula 102 by the pulse generation system 1302 ensures that the patient's circulatory system is sufficiently perfused while simultaneously preventing injury to the patient.

As previously discussed, at least a portion of the fluid flowing through the pulse generator 136 is provided to the sensor block 124 along the sensor block inlet line 146. The sensor block 124 includes a housing 126 configured to receive and contain fresh perfusate flowing from the pulse generator 136 and a plurality of sensor probes 128 positioned within the housing 126 of the sensor block 124. The housing 126 of the sensor block 124 is fluidly coupled to the perfusate reservoir 104 along a sensor block outlet line 148, and fresh perfusate flowing through the sensor block 124 is provided to the perfusate reservoir 104 along the sensor block outlet line 148.

The sensor probes 128 are configured to measure certain characteristics of the fresh perfusate inside the housing 126 including, but not limited to, pH, dissolved oxygen concentration, dissolved nitrogen concentration, dissolved carbon dioxide concentration, viscosity, redox potential, temperature, conductivity, and oxygen carrying capacity. As previously described, the oxygenator 116, the heat exchanger 118, and the gas mixer 120 are controlled based on the characteristics measured by the sensor probes 128 in order to optimize the temperature, O₂ level, and gas composition of the perfusate. In some implementations, an alpha stat approach is used to control the concentration of gasses with the fresh perfusate and the pH of the fresh perfusate.

A stopcock 155 is positioned along the perfusion line 144 downstream of the resistance valve 152. An operator of the system 100 can use the stopcock 155 to sample fluid from the perfusion line 144, for example, to test one of more properties of the perfusate being provided to the cannula 102. In addition, the stopcock 155 can be operated in order to control the flow of air along the perfusion line during initial set up and calibration of the system 100. For example, during initial calibration of the pressure sensor 141, pressurized air is provided to the perfusion line along the air calibration line 163 through stopcock 155, and the valves 152, 159 along the perfusion line 144 are both closed to generate a stable air pressure along the perfusion line 144 between the valves 152, 159, and the pressure sensor 141 can be calibrated based on the measured pressure.

A solenoid valve 159 is positioned along the perfusion line 144 downstream of the second pressure sensor 141 and the second stopcock 155. The solenoid 159 is configured to control fluid flow from the perfusion line 144 into the cannula 102. For example, during perfusion of a patient coupled to the cannula 102, the solenoid 159 is opened to allow fluid within the perfusion line 144 flow through the cannula 102 and into a blood vessel of the patient coupled to the cannula 102. The solenoid 159 is closed during calibration of the pressure sensors 140, 141 and the flow sensors 142 to prevent fluid along the perfusion line 144 from flowing out of the cannula 102.

The cannula 102 of the perfusion system 100 is configured to be inserted into an arterial blood vessel of a patient in order to fluidly couple the patient's circulatory system to the perfusion line 144. In some implementations, the cannula is 15 French diameter cannula. In some implementations, the cannula 102 is configured to minimize resistance and optimize fluid dynamics of perfusate flowing through the cannula 102. In some implementations, the cannula 102 is configured so that resistance of perfusate flowing through the cannula 102 is within a predetermined range. In some implementations, the cannula 102 includes an integrated pressure sensor configured to measure a pressure of fluid flowing through the cannula 102, and the pulse generation system 1302 is controlled based partly on the pressure sensor data generated by the integrated pressure sensor of the cannula 102. In some implementations, the cannula 102 is inserted into the blood vessel of patient together with an endovascular sheath.

Still referring to FIG. 1, the perfusate reservoir 104 includes a reservoir housing 103 that is configured to receive fresh perfusate from the sensor block 124 along the sensor block outlet line 148. The reservoir housing 103 is a watertight container that can be formed of any suitable material including, but not limited to, plastic, metal, or glass. In some implementations, the perfusate reservoir 104 includes a filter bag positioned within the reservoir housing 103 to filter the fluid flowing into the perfusate reservoir 104. In some implementations, the filter bag is formed of a 0.22 μm filter.

The perfusion system 100 includes a syringe pump manifold 167 that is configured to support one or more syringes and a stepper motor that is configured to control movement of a respective plunger of each of the syringes contained within the manifold 167. The syringe pump manifold 167 is fluidly coupled to the perfusate reservoir 104 along an input line 169. Each of the syringes contained within the syringe pump manifold is filled with a therapeutic compound, such as an oxygen carrier compound, and the syringe pump manifold 167 is controlled during the perfusion process to add the therapeutic compound to the fresh perfusate in the perfusate reservoir 104 at a predetermined rate. The therapeutic compound provided to the perfusate reservoir 104 by the syringe pump manifold 167 is subsequently provided to the circulatory system of the patient by delivering the perfusate containing the therapeutic compound to the patient through the cannula 102 coupled to the patient. The syringe pump manifold 167 is configured to introduce therapeutic compounds into the perfusate in the reservoir 104 at a controlled rate to achieve the desired therapeutic effect while maintaining target pressure and flow rate along the perfusion line 144 and limiting changes of viscosity of the perfusate. The therapeutic compounds that can be provided to the perfusate reservoir using the syringe pump manifold include, but are not limited to, heparin, verapamil, penicillin, streptomycin, adenosine, glucose, vasopressors (e.g., norepinephrine (e.g., Levophed), phenylephrine, epinephrine), antifibrinolytic agents (e.g., tranexamic acid (TXA)), fluid resuscitation agents (e.g., saline solutions, albumin), blood products (e.g., packed red blood cells (PRBCs), fresh frozen plasma (FFP), platelets), analgesics (e.g., morphine, fentanyl), and anesthetics (ketamine).

In some implementations, the syringe pump manifold 167 is configured to control the amount of therapeutic compound added to the perfusate in the reservoir 104 based on vital sign data generated by the vital sign sensors 115. Based on the data generated by the vital sign sensors 115, the syringe pump manifold 167 can be controlled to add one or more therapeutic compounds to the perfusate reservoir to minimize vascular injuries, optimize oncotic pressure of the perfusate, limit thrombic complications, and improve the hemodynamic, vascular, and metabolic support provided to the patient by the perfusion system.

For example, in some implementations, the vital sign sensors 115 can measure the oxygen saturation of the patient throughout the perfusion process and, based on the measured oxygen saturation of the patient, the syringe pump manifold 167 can be controlled to introduce an enhanced oxygen carrier compound into the perfusate reservoir 104 in order to increase the oxygen saturation of the patient. In some implementations, the syringe pump manifold is 100% pure erythrocruorin derived from Lumbricus terrestris (“LtEc”) to provide enhanced oxygen carrying capacity to the perfusate. The process for isolating and extracting LtEc from Lumbricus terrestris can be optimized and scaled for large-scale manufacturing of LtEc for use by the perfusion system 100. In some implementations, a tangential flow filtration system can be used to isolate, extract, and purify the LtEc compound. The LtEc enhanced oxygen carrier compound administered to the patient using the perfusate system 100 can be manufactured to achieve particular purity and sterility requirements for administration to a human.

In some implementations, the purity of the LtEc enhanced oxygen carrier compound administered by the syringe pump manifold 167 is less than 0.5 EU/ml. In some implementations, the LtEc enhanced oxygen carrier compound administered to the patient using the perfusate system 100 has approximately 40 times the oxygen carrying capacity as hemoglobin.

Referring to FIG. 1, the perfusate system 100 includes a hematocrit sensor 180 fluidly coupled to the perfusion line 144 downstream of the second pressure sensor 141 and the solenoid valve 159. The syringe pump manifold 167 can additionally or alternatively be controlled to deliver one or more therapeutic compounds to the perfusate reservoir 104 based on hematocrit values of the patient's blood measured by the hematocrit sensor 180. For example, during the perfusion process, the solenoid valve 159 can be closed and the pulse generation system 1302 can be controlled to temporarily stop providing perfusate to the perfusion line 144. As a result, blood flows from the patient through the cannula 102 and along the perfusion line 144 to the hematocrit sensor 180, and the hematocrit sensor 180 measure the hematocrit value of the patient's blood. Once the hematocrit value is measured using the hematocrit sensor 180, the solenoid valve 159 can be closed and the pulse generation system 1302 can be controlled to resume providing perfusate to the perfusion line 144 in order to resume perfusion of the patient's circulatory system. Based on the hematocrit value measured by the hematocrit sensor 180, hemodilution of the patient's blood can be detected. In response to detecting hemodilution of the patient's blood, the syringe pump manifold 167 is controlled to deliver an enhanced oxygen carrier compound into the perfusate reservoir 104 for perfusion to the patient to treat the hemodilution.

In some implementations, the therapeutic compound delivered using the syringe pump manifold 167 is a super plasma expander configured to reduce an overall viscosity of the patient's blood. For example, based on signals received from the vital sign sensors 115, such as the patient blood pressure and arterial blood flow measurements, the syringe pump manifold 167 can be controlled to deliver a super plasma expander compound to the perfusate reservoir 104 at a rate determined based on the vital sign sensor 115 data. As a result of delivering a super plasma expander to the perfusate using the syringe pump manifold 167 and flowing the perfusate to the patient through the cannula 102, the patient's arterial flow rate and capillary density is increased.

In some implementations, the syringe pump manifold 167 is configured to deliver polyethylene glycol-20k (PEG-20k) into the perfusate reservoir 104 in order enhance low-volume resuscitation provided by the perfusion system 100 to a patient experiencing a hemorrhage. In some implementations, the syringe pump manifold 167 is configured to deliver Factor XIa inhibitor into the perfusate reservoir 104 to reduce coagulation and prevent vascular injury.

The perfusate used in the perfusion system 100 is an acellular solution configured to restore intravascular volume and maintain perfusion to organs when administered to a patient experiencing a hemorrhage. In addition, the perfusate provided to the patient using the perfusion system 100 is configured to minimize hemodilution and maintain the oxygen transfer rate of the patient's blood, as determined based on signals generated by the vital sign sensors 115 and/or the hematocrit sensor 180. Testing and optimization of the perfusate can be performed to optimize the oncotic pressure, oxygen carrying capacity, viscosity, and electrolytic composition of the perfusate. In addition, testing and optimization of the perfusate can be performed to determine and control the immune-oxidative stress caused by providing the perfusate to a patient. The oncotic pressure of the perfusate is configured to prevent extravasation of the perfusate from the patient's blood vessels. In some implementations, when perfused to a patient using the perfusion system 100, the perfusate maintains an oxygen transfer rate greater than 75 ml/min transfer rate per volume loss.

In some implementations, the perfusate includes an enhanced oxygen carrier compounds, such as the LtEc enhanced oxygen carrier compound described herein. In some implementations, the amount of enhanced oxygen carrier compound contained within the perfusate enables the patient to maintain a target oxygen partial pressure of oxygen when treated with the perfusate. In some implementations, the perfusate includes an amount of enhanced oxygen carrier compound sufficient to provide the perfusate with normal physiological levels of oxygen carrying capacity in order to prevent hemodilution when a patient is treated with the perfusate.

In some implementations, the perfusate solution is lyophilized during manufacturing to generate a powdered, shelf-stable perfusate compound that is configured to be reconstituted by adding a liquid to the powdered perfusate to liquify and resuspend the perfusate compound. A lyophilization instrument can be used to lyophilize the perfusate solution, and the lyophilized perfusate can be assessed based on moisture, mass conversation, and stability of perfusate species components. Shelf-stable, lyophilized perfusate enables easier transportation of the perfusate and is particularly beneficial for treating patient's experiencing a hemorrhage in a battlefield setting. The amount of liquid added to the lyophilized perfusate powder is selected to provide the liquified perfusate with specific perfusate pH, component concentration, osmolarity, viscosity, and oncotic pressure values. In addition, the amount of liquid added to the lyophilized perfusate powder enables the perfusate solution to be able to provide sufficient euvolemic control when whole body perfusion is conducted using the perfusate. The amount of liquid added to the lyophilized perfusate powder is also selected to ensure maintenance of the oxygen transfer rate and prevent hemodilution during whole body perfusion.

Referring to FIG. 1, the perfusate system includes a computer system 188 that is communicably coupled to the electrical components of the perfusate system 100 including, but not limited to pump 114, the vital sign sensors 115, the heat exchanger 118, the oxygenator 116, the gas mixer 120, the electronic pressure regulator 162, the sensor probes 128, the pressure sensors 140, 141, the flow sensors 130, 142, the resistance valve 152, 159, the syringe pump manifold 167, and the solenoid valves 158, 159. In some implementations, the computer system 188 can be in wireless or wired communication with the electronic components of the perfusate system 100. In some implementations, a display 189 of the computing device 188 is configured to display data generated by the perfusate system 100. For example, sensor data generated by the sensor probes 128 can be transmitted to the computer system 188 in real-time, and the display 189 can be configured to display sensor data received from the sensor probes 128. In some implementations, the data generated by the perfusate system 100 (e.g., the sensor data generated by the sensor block 124 and the vital sign sensors 115) is transmitted in real-time to the computer system 188, and the computer system 188 is configured to transmit the data generated by the perfusate system 100 to a remote database for storage. The computer system 188 is also configured to communicably couple and controls the electronic components of the perfusate system 100 to perform the perfusion process as described herein. For example, the computer system 188 is configured to receive data from the vital sign sensors, the pressure sensor 141, and the flow sensor 142, and based on the sensor data, control the electronic pressure regulator 162 to adjust the pulsatile flow provided by the pulse generator 136 along the perfusion line 144.

An example process of performing perfusion of a patient's circulatory system using the system 100 will now be described with reference to FIG. 1.

Prior to performing a perfusion treatment, the perfusate reservoir 104 is filled with perfusate solution. In some implementations, the perfusate solution is lyophilized during manufacturing to generate a powdered, shelf-stable perfusate compound, and filling the perfusate reservoir 104 with perfusate involves combining the powdered perfusate compound to a liquid to liquify and resuspend the perfusate compound.

Once the perfusate reservoir 104 is filled with perfusate, the vital sign sensors 115, flow sensors 130, 142, and pressure sensors 140, 141 of the system 100 are calibrated and the fluid line 112 and the perfusion line 144 are flooded with perfusate from the reservoir 104 to remove any air from the lines 112, 144.

Once the sensors 115, 130, 140, 141, 142 are calibrated and the fluid lines 112, 144 of the arterial circuit 106 are flooded with perfusate, the cannula 102 is inserted into an arterial blood vessel of a patient experiencing a hemorrhage and the vital sign sensors 115 are coupled to the patient to begin measuring the vital signs of the patient.

Once the cannula 102 is inserted into the arterial blood vessel of the patient, the flow pump 114 is controlled to flow perfusate through the oxygenator along the arterial fluid line 112 to the pulse generator 136. As previously described, due to the relative positioning of the pulse generator 136, the cannula 102, and the sensor block 124, and the structure of the pulse generator 136, all of the perfusate provided to the pulse generator 136 from the arterial fluid line 112 passively flows through the sensor block inlet line 146 when the diaphragm 214 of the pulse generator 136 is in an unflexed (“neutral”) position.

As perfusate passes through the sensor block 124, sensor probes 128 of the sensor block 124 measure one or more characteristic of the fresh perfusate inside the housing 126, including, but not limited to, pH, dissolved oxygen concentration, dissolved nitrogen concentration, dissolved carbon dioxide concentration, viscosity, temperature, conductivity, and oxygen carrying capacity. Based on the characteristics measured by the sensor probes 128, the oxygenator 116, heat exchanger 118, and gas mixer 120 are controlled in order to optimize the temperature, O₂ level, and gas composition of the perfusate.

As previously discussed, the heat exchanger 118 is configured to adjust the temperature of the perfusate flowing through the heat exchanger 118 based on the temperature of the perfusate detected by the sensor block 124. The temperature of the perfusate is adjusted by the heat exchanger 118 to maintain a target temperature for the perfusate, as detected based on the sensor probes 128 in the sensor block 124. In some implementations, the target temperature is 37° C. In addition, at the beginning of a perfusion procedure, the O₂ concentration of the perfusate is controlled by the gas mixer 120 to maintain a target partial pressure of O₂ in the perfusate (pO₂), as detected based on the sensor probes 128 in the sensor block 124. In some implementations, the target partial pressure of O₂ in the perfusate is 200-250 mmHg.

While the temperature and O₂ concentration of the perfusate are being controlled by the heat exchanger 118 and the gas mixer 120, the pulse generation system 1302 is simultaneously controlled to adjust the pressure and flow rate of the perfusate provided along the perfusion line 144 to the cannula 102. For example, as previously discussed, due to the relative positioning of the pulse generator 136, the cannula 102, and the sensor block 124, and the structure of the pulse generator 136, none of the perfusate provided to the pulse generator 136 passively flows through the perfusion line 144 to the cannula 102 when the diaphragm 214 of the pulse generator 136 is in an unflexed (“neutral”) position. Therefore, in order to provide pulsatile flow of perfusate to the cannula 102 along the perfusion line 144, the air supply system 138 is controlled to provide pulsatile, pressurized air streams to the pulse generator 136 in order to cause the diaphragm 214 of the pulse generator 136 to flex downwards towards the fluid chamber 236, which causes the diaphragm 214 to cover the openings 210 in the fluid chamber 236 of the pulse generator 136 and increase the pressure within the interior 212 of the pulse generator 136. As result of the increased pressure caused by the flexing of the diaphragm 214, at least a portion of the fresh perfusate contained within the interior 212 of the pulse generator is forced through the perfusion line outlet 208 of the pulse generator 136 and along the perfusion line 144 to the cannula 102.

During the perfusion process, the pulse generation system 1302 is controlled based on data generated by the vital sign sensors 115, the pressure sensor 141, and the flow sensor 142. For example, the vital sign sensors 115 continuously measure the patient's blood pressure in real time throughout the perfusion treatment and, in order to control the blood pressure of the patient within a normal physiological range, the pulse generation system 1302 is controlled to increase or decrease the pressure along the perfusion line 144, as measured by the pressure sensor 141, based on the blood pressure of the patient detected by one or more of the vital sign sensors 115. Based on the blood pressure of the patient measured by the vital sign sensors 115, the pulse generator 136 is automatically controlled to adjust the pressure along the perfusion line 144, as detected by the pressure sensor 141, until the blood pressure of the patient detected by the vital sign sensors 115 is within a normal physiological range.

In order to control the blood pressure of the patient within a normal physiological range during a hemorrhage, the pulse generation system 1302 is also controlled to automatically increase or decrease the flow rate of perfusate along the perfusion line 144, as measured by the flow sensor 142, based on the blood pressure and arterial blood flow of the patient detected by one or more of the vital sign sensors 115. For example, the vital sign sensors 115 continuously measure the patient's blood pressure and arterial flow rate in real time throughout the perfusion treatment. Based on the blood pressure and arterial flow rate of the patient measured by the vital sign sensors 115, the pulse generation system 1302 dynamically controls the flow rate of perfusate along the perfusion line 144, as detected by the flow sensor 142, until the blood pressure and arterial blood flow of the patient detected by the vital sign sensors 115 are within a normal physiological range.

Throughout the perfusion process, the pulse generation system 1302 is also controlled based on signals generated by the pressure sensor 141 and the flow sensor 142 to maintain the pressure and flow rate along the perfusion line 144 (as detected by pressure sensor 141 and flow sensor 142) within respective predetermined ranges. By controlling the pressure and flow rate along the perfusion line 144 to each be maintained within a predetermined range, responsive to the level of resistance of the patient's arterial system measured by the flow sensor 142 and pressure sensor 141, the perfusion of the patient's circulatory system is improved and the risk of vascular injury during perfusion is reduced.

Perfusate flowing along the perfusion line 144 is flows through the cannula 102 and into the arterial blood vessel of the patient coupled the cannula 102, and perfuses the entire circulatory system of the patient, including the arteries, capillaries, and veins of the patient. The perfusion system 100 is operated to continue perfusing the patient's circulatory system until the hemorrhage being experienced by the patient is treated or otherwise controlled.

During the perfusion process, the syringe pump manifold 167 is controlled to introduce one or more therapeutic compounds into the perfusate reservoir 104, which are then provided to the patient's circulatory system through the perfusate. As previously described, the syringe pump manifold 167 is configured to control the amount of therapeutic compound added to the perfusate in the reservoir 104 based on vital sign data generated by the vital sign sensors 115. Based on the data generated by the vital sign sensors 115, the syringe pump manifold 167 can be controlled to add one or more therapeutic compounds to the perfusate reservoir to minimize vascular injuries, optimize oncotic pressure of the perfusate, limit thrombic complications, and improve the hemodynamic, vascular, and metabolic support provided to the patient.

Once the perfusion process is complete, the pump 114 is controlled to stop pumping the perfusate through the system 100, the valve 159 along the perfusion line 144 is closed, and the cannula 102 is removed from the patient to disconnect the patient from the perfusion system 100.

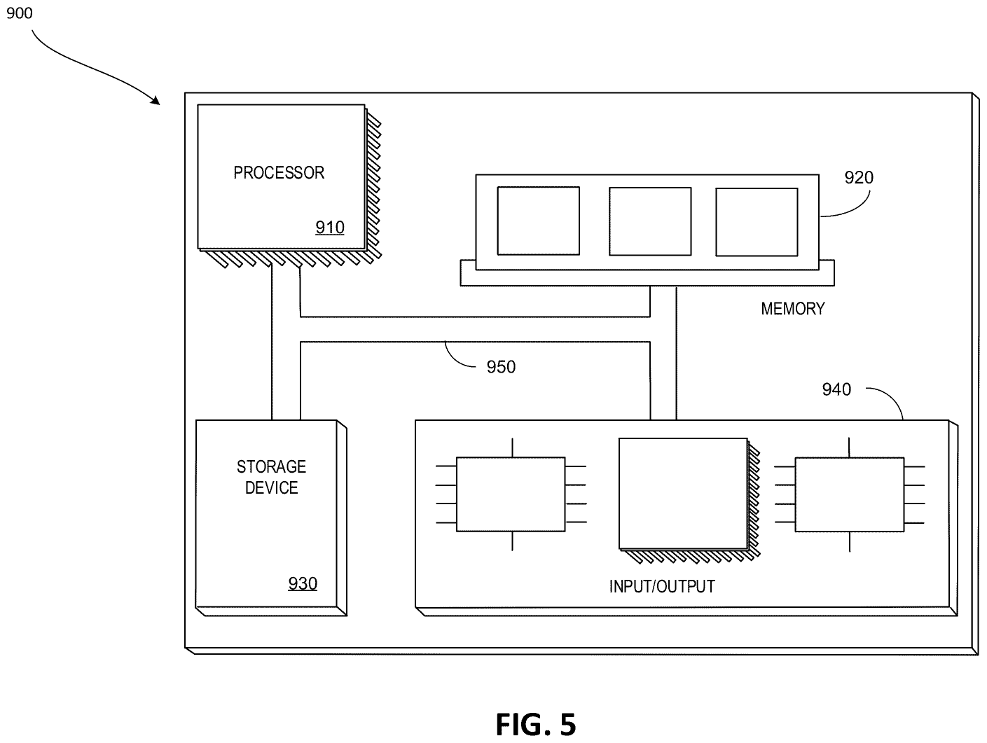

FIG. 5 is a block diagram of an example computer system 900. For example, referring to FIG. 1, the computer system 188 of the perfusate system 100 could be an example of the system 900 described here. The system 900 includes a processor 910, a memory 920, a storage device 930, and an input/output interface 940. Each of the components 910, 920, 930, and 940 can be interconnected, for example, using a system bus 950. The processor 910 is capable of processing instructions for execution within the system 900. The processor 910 can be a single-threaded processor, a multi-threaded processor, or a quantum computer. The processor 910 is capable of processing instructions stored in the memory 920 or on the storage device 930. The processor 910 may execute operations such as receiving signals from one or more sensors (e.g., the vital sign sensors 115, sensor probes 128, the pressure sensors 140, 141, the flow sensors 130, 142, shown in FIG. 1) and controlling or more elements of the system (e.g., the solenoid valves 158, 159 of FIG. 1) based on the received signals.

The memory 920 stores information within the system 900. In some implementations, the memory 920 is a computer-readable medium. The memory 920 can, for example, be a volatile memory unit or a non-volatile memory unit. In some implementations, the memory 920 stores a data structure. In some implementations, multiple data structures are used.

The storage device 930 is capable of providing mass storage for the system 900. In some implementations, the storage device 930 is a non-transitory computer-readable medium. The storage device 930 can include, for example, a hard disk device, an optical disk device, a solid-date drive, a flash drive, magnetic tape, or some other large capacity storage device. The storage device 930 may alternatively be a cloud storage device, e.g., a logical storage device including multiple physical storage devices distributed on a network and accessed using a network.

The input/output interface 940 provides input/output operations for the system 900. In some implementations, the input/output interface 940 includes one or more of network interface devices (e.g., an Ethernet card), a serial communication device (e.g., an RS-232 10 port), and/or a wireless interface device (e.g., an 802.11 card, a 3G wireless modem, or a 4G wireless modem). In some implementations, the input/output device includes driver devices configured to receive input data and send output data to other input/output devices, e.g., keyboard, printer and display devices (e.g., display device 189 of FIG. 1). In some implementations, mobile computing devices, mobile communication devices, and other devices are used.

In some implementations, the system 900 is a microcontroller. A microcontroller is a device that contains multiple elements of a computer system in a single electronics package. For example, the single electronics package could contain the processor 910, the memory 920, the storage device 930, and input/output interfaces 940.

Although an example processing system has been described in FIG. 5, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “computer system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

A number of embodiments have been described. Nevertheless, it will be understood that various modifications may be made without departing from the spirit and scope of the disclosure.

For example, while the pump 114 has been described as being a peristaltic pump, in some implementations, other types of pumps can be used. In some implementations, the pump 114 is a roller pump or a centrifugal pump.

While the perfusion system 100 has been described as including a syringe pump manifold 167 with two or more syringes filled with a pharmaceutical compound, in some implementations, the perfusion system 100 includes a single syringe pump with a single syringe filled with a therapeutic compound.

While the system 100 has been depicted as including pressure sensors 140, 141, a flow sensor 142, and a hematocrit sensor 180 coupled to the perfusion line 144, one or more additional sensors can be coupled to the perfusion line 144. For example, in some implementations, the system 100 includes a conductivity sensor coupled to the perfusion line 144 that is configured to measure the conductivity of the perfusate flowing along the perfusion line 144 and/or the conductivity of the patient's blood. In some implementations, the system 100 includes a spectrophotometric sensor coupled to the perfusion line 144 that is configured to measure the iron or hemoglobin levels of the patient's blood.

While the lid 218, the central housing 202, and the base 220 of the pulse generator 136 have been described as being attached to one another using a set of bolts 224 and nuts 228, other types of mechanical fasteners can be used to couple the lid 218, central housing 202, and base 220. For example, in some implementations, the lid 218, the central housing 202, and the base 220 are attached to one another using one or more of bolts, rivets, pins, or adhesive.

While the computer system 188 has been depicted as including a single computing device, in some implementations, the computing system 188 includes multiple computing devices or processors (2, 3, 4, etc.) in communication with the various electronic components of the perfusion system 100 and configured to control operation of one or more electronic components of the perfusion system 100. Each of the computing devices of the computer system 188 may be in wired or wireless communication with one or more electronic components of the perfusion system 100. In addition, one or more of the computing devices of the computer system 188 can be a remote computing device that is located at a different, remote location from the perfusion system 100.

While the cannula 102 has been depicted as a single lumen cannula, in some implementations, the cannula 102 is a double lumen cannula with a first lumen configured to flow perfusate from the perfusion line 144 into the patient and a second lumen configured to flow blood from the patient into a portion of the perfusion line (e.g., to the hematocrit sensor 180).

In addition, while the cannula 102 has been described as being configured to be inserted into an arterial blood vessel of the patient, in some implementations, the cannula 102 is configured to be inserted into a venous blood vessel of the patient, and the whole body perfusion is performed by flowing perfusate into the venous blood vessel fluidly coupled to the cannula.

While the pressure along the perfusion line 144 has been described as being dynamically controlled based on vital sign data generated by the vital sign sensors 115, in some implementations, the pulse generation system 1302 is controlled based on signals generated by the pressure sensor 141 in order to provide pulsatile flow along the perfusion line 144 sufficient to maintain the predetermined pressure along the perfusion line 144 as detected by the pressure sensor 141. In some implementations, the pulse generation system 1302 is configured to maintain a pressure along the perfusion line 144 that corresponds to normal physiological arterial blood pressure or slightly above normal physiological arterial blood pressure, as measured by the pressure sensor 141.

Similarly, while the flow rate along the perfusion line 144 generated by the pulse generation system 1302 has been described as being dynamically controlled based on vital sign data generated by the vital sign sensors 115, in some implementations, the pulse generation system 1302 is controlled based on signals generated by the flow sensor 142 in order to provide pulsatile flow along the perfusion line 144 sufficient to maintain the predetermined flow rate along the perfusion line 144 as detected by the flow sensor 142. In some implementations, the pulse generation system 1302 is configured to maintain a flow rates that corresponds to normal physiological blood flow rates along the perfusion line 144, as measured by the flow sensor 142.

While the blood pressure of the patient has been described as being measured using one or more vital sign sensors 115, in some implementations, the patient's blood pressure can be detected based on the flow rate and pressure measured along the perfusion line 144 using the flow sensor 142 and pressure sensor 141 along the perfusion line 144. For example, during perfusion treatment, the resistance valve 152 along the perfusion line 144 can be closed, and the patient's arterial pressure can be determined based on the pressure measured along the perfusion line 144 by the pressure sensor 141 while the resistance valve 152 is closed. The intraarterial pressure measurements generated by the pressure sensor 141 can be used to determine intraluminal and/or aortic pressure and blood flow of the patient. In addition, the data generated by the pressure sensor 141 and flow sensor 142 along the perfusion line 144 can be analyzed to measure the reflected pressure wave generated along the perfusion line by the patient's arterial resistance, which can be used to determine whether the patient's vasculature is atherosclerotic.

In some implementations, a machine learning model can be trained to detect the blood pressure of a mammal being treated with the system 100 based only on the data generated by the pressure sensor 141 and flow sensor 142 along the perfusion line 144. For example, a series of experiments can be conducted in which the blood volume of a mammalian test subject (such as a pig) is controlled to simulate a hemorrhage condition and the test subject can be treated using the perfusion system, and data generated during these experiments can be used to train a machine learning model to determine the vital signs, including blood pressure and blood flow, of a patient based on the data generated by the perfusion line sensors 141, 142.

The arterial blood pressure and the arterial blood flow rate of the test subject are measured throughout the test using vital sign sensors 115, such as a blood pressure sensor and blood flow sensor. As previously discussed, FIG. depicts example vital sign sensor data depicting the arterial blood pressure 602 and arterial blood flow rate 604 of a pig that was tested with a simulated hemorrhage and treated with circulatory system perfusion using the perfusion system 100 depicted in FIG. 1. After a predetermined amount of blood was removed from the pig, the cannula 102 of the perfusion system 100 was inserted into an arterial blood vessel of the pig, and perfusion of the circulatory system of the pig was conducted using the perfusion system 100, as described herein. As can be seen in FIG. 3, perfusion of the circulatory system of the pig with perfusate using the perfusion system 100 resulted in the arterial blood pressure 602 and arterial blood flow 604 of the pig to recover to normal levels. FIG. 4 includes data depicting the pressure 706 along the perfusion line 144 throughout perfusion as measured by pressure sensor 141 and the flow rate 708 along the perfusion line 144 throughout perfusion as measured by flow sensor 142. During testing, the pulse generator 136 of the system 100 was dynamically controlled to provide a pulsatile flow of perfusate along the perfusion line 144 and through the cannula at a pressure 706 and flow rate 708 that was sufficient to return the arterial blood pressure 602 and arterial blood flow rate 604 to normal, pre-hemorrhage levels, as depicted in FIG. 4.

The experiment described above can be conducted several times, and the blood pressure data 602, blood flow rate data 604, perfusion line pressure data 706, perfusion line flow rate data 708 for each experiment can be provided to a machine learning model as training data. Based on the training data generated during the hemorrhage experiments, the machine learning model can be trained to determine the arterial blood pressure and the arterial blood flow rate of a patient treated with the perfusion system 100 in real time based on the pressure and the flow rate measured along the perfusion line 144 by the pressure sensor 141 and flow sensor 142, respectively. For example, during perfusion of a patient using the perfusion system, data generated by the pressure sensor 141 and flow sensor 142 can be provided to the trained machine learning model, which can use the perfusion line sensor data to determine the patient's arterial blood pressure and arterial blood flow rate in real time throughout the perfusion treatment. As a result, the pulse generation system 1302 can be automatically and dynamically controlled to restore and maintain the blood pressure and blood flow rate of the patient within a normal physiological level while the patient is experiencing a hemorrhage based on the data generated by the pressure sensor 141 and flow sensor 142 along the perfusion line without requiring the use of external vital sign sensors. By controlling the pulse generator 136 without requiring the use of external vital sign sensors, such as sensor 115 of FIG. 1, the perfusion system can be more compact and efficiently operated.

In addition, while the syringe pump manifold 167 has been described as being automatically controlled based on data generated by the vital sign sensors, in some implementations, the syringe pump manifold 167 is automatically controlled by a machine learning model. For example, the data generated by the vital sign sensors 115, the pressure sensor 141, and the flow sensor 142 can be provided to a trained machine learning model in order to predict one or more characteristics about the patient's circulatory system, and the syringe pump manifold 167 can be automatically controlled by on the characteristics determined by the machine learning model. Data generated by the vital sign sensors 115, the pressure sensor 141, and the flow sensor 142 collected throughout numerous perfusion processes on respective patients, a machine learning model can be trained to identify data generated by the pressure sensor 141 and/or the flow sensor 142 corresponding to particular vital signs of the patient, as well as other cardiac conditions of the patient, such as cardiac output of the patient's heart. Once the machine learning model has been trained, the data generated by the vital sign sensors 115, the pressure sensor 141, and/or the flow sensor 142 can be provided to the trained machine learning model to identify certain cardiac conditions of the patient and predict certain responses or reactions relating to the administration of one or more therapeutic compounds to the patient. As a result, the trained machine learning model can be used to automatically control the syringe pump manifold 167 to automatically administer one or more therapeutic substances to the patient throughout the perfusion treatment in order to minimize vascular injuries, optimize oncotic pressure of the perfusate, limit thrombic complications, and improve the hemodynamic, vascular, and metabolic support provided to the patient during a hemorrhage event.

While the machine learning model has been described as being trained using sensor data generated during in vivo experiments, the machine learning model can additionally or alternatively be trained using data generated by performing in vitro experiments with the perfusion system 100.

In addition, while the perfusion treatment has been described as being performed and controlled automatically based on data generated by one or more sensors 115, 128, 130, 141, 142, 180, in some implementations, a user can control or alter one or more steps of the perfusion process. For example, the computer system 188 can display a user interface on the display 189 to enable a user to control one or more features of the perfusion system (e.g., to start or stop perfusion, adjust the pressure or flow rate along perfusion line 144, control the syringe pump manifold 167 to add therapeutics to the perfusate reservoir 104, etc.).

While the perfusate system 100 has been described as being used to perfuse the circulatory system of a human experiencing a hemorrhage, the perfusion system 100 can be used to perfuse the circulatory system of other mammals, such as pigs. In some implementations, an alternate cannula 102 with a modified structure is used in order to perfuse non-human circulatory systems with the perfusion system 100. In addition, the perfusate used in the perfusate system 100 may be altered in order to more effectively perfuse non-human mammalian circulatory systems.

While the perfusion system 100 has been described as being used to treat a patient experiencing a hemorrhage, in some implementations, the perfusion system 100 can be used to provide whole body perfusion to sustain a patient experiencing one or more injuries or conditions in addition to a hemorrhage (i.e., a “polytrauma” patient). For example, the perfusion system 100 can be used to treat a patient simultaneously experiencing a hemorrhage and a lung injury.

In some implementations, the perfusate used by the perfusion system 100 is altered or modify based on the injuries or conditions of the patient being treated in order to provide optimized hemodynamic, vascular, oxygenation, and metabolic support and increase survival times. For example, the oxygen carrier profile of the perfusate can be adjusted to better support the non-hemorrhage injuries or conditions being experienced by the patient. In addition, one or more therapeutic compounds can be added to the perfusate, either during manufacturing of the perfusate or during treatment (e.g., using the syringe pump manifold) in order to better address potential thrombotic complications and other potential vascular related injuries arising due to the non-hemorrhage injuries or conditions of the patient being treated with the perfusion system 100.

One or more additional or alternative vital sign sensors 115 and corresponding vital sign measurements may be included in the perfusion system 100 in order to better monitor and respond to non-hemorrhage injuries or conditions of the patient. Similar to the testing and machine learning model training described above with reference to FIGS. 3 and 4, testing of the perfusion system 100 can be performed to determine pressure and flow rate values along the perfusion line 144 optimized to provide support and euvolemic control and blood flow rate control to sustain a patient experiencing one or more non-hemorrhage injuries or conditions. In addition, based on data generated during testing, the machine learning model used to control the perfusion system 100 can be modified to automatically control the perfusion system 100 to provide optimized support and euvolemic control and blood flow rate control to a patient experiencing non-hemorrhage injuries or conditions.

In addition, while the perfusion system 100 has been as being used to treat a patient experiencing a hemorrhage, in some implementations, the perfusion system can be used to provide hemodynamic, vascular, oxygenation, and metabolic support to a patient that is not experiencing a hemorrhage, but is instead experiencing one or more non-hemorrhage injuries or conditions. Whole body perfusion using the perfusion system 100 can be configured to provide hemodynamic, vascular, oxygenation, and metabolic support and increase survival times for patients experiencing one or more non-hemorrhage injuries or conditions including, but not limited to, heart attack, anemia, ischemic stroke, peripheral vascular disease, trauma, respiratory failure, and other conditions that require oxygenation and anti-coagulation support. As previously described, one or more components and/or operations of the perfusion system 100 can be modified to better support a patient experiencing one or more non-hemorrhage injuries or conditions.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A perfusion system for treatment of a patient experiencing at least one medical condition, the system comprising:
a reservoir configured to receive perfusate;
a cannula configured to fluidly couple the reservoir to an arterial blood vessel of a mammal through a single entry point;
a fluid line fluidly coupling the reservoir to the cannula;
a pressure sensor configured to measure pressure along the fluid line;
a flow sensor configured to measure a flow rate along the fluid line; and
a pulse generation system comprising a pulse generator, the pulse generation system configured to generate pulsatile flow of perfusate from the reservoir to the cannula along the fluid line based on one or more signals generated by at least one of the pressure sensor or the flow sensor,
wherein the perfusion system is configured to perfuse an entire circulatory system of the mammal through the single entry point to treat a patient experiencing at least one medical condition.

2. The system of claim 1, wherein the pulse generation system is controlled based on an internal resistance of the circulatory system of the mammal detected by the pressure sensor.

3. The system of claim 1, further comprising at least one vital sign sensor configured to measure at least one of blood pressure, blood oxygen concentration, heart rate, or core temperature wherein the pulse generation system is controlled based on one or more signals generated by the at least one vital sign sensor.

4. The system of claim 3, wherein:
the system further comprises at least one of (i) a heat exchanger configured to control a composition of gasses within the perfusate or (ii) a gas mixer configured to control a composition of gasses within the perfusate; and
at least one of (i) the heat exchanger is controlled based at least partly on one more signals generated by the at least one vital sign sensor or (ii) the gas mixer is controlled based at least partly on one more signals generated by the at least one vital sign sensor.

5. The system of claim 1, wherein the mammal is a human.

6. The system of claim 1, further comprising a syringe pump manifold fluidly coupled to reservoir, wherein the syringe pump manifold is configured to introduce one or more substances into the perfusate in the reservoir.

7. The system of claim 6, wherein:
the system further comprises at least one vital sign sensor configured to measure one or more vital signs of the mammal; and
the syringe pump manifold is configured to introduce the one or more substances into the perfusate in the reservoir based on one or more signals generated by one the at least one vital sign sensor.

8. The system of claim 6, wherein the one or more substances comprise an oxygen carrier substance.

9. The system of claim 8, wherein the oxygen carrier substance comprises erythrocruorin derived from Lumbricus terrestris.

10. The system of claim 6, wherein:
the system further comprises a hematocrit sensor fluidly coupled to the fluid line downstream of the pulse generator; and
the syringe pump manifold is configured to introduce the one or more substances into the perfusate in the reservoir based on one or more signals generated by the hematocrit sensor.

11. The system of claim 1, further comprising a pump fluidly coupled to the reservoir and to the pulse generator, wherein the pump is configured to flow perfusate from the reservoir to the pulse generator.

12. The system of claim 1, wherein the perfusion system is configured to increase a blood pressure of the mammal.

13. The system of claim 1, wherein the perfusate is an acellular composition.

14. The system of claim 1, further comprising an air supply system fluidly coupled the pulse generation system, the air supply system comprising:
an air source;
an electronic pressure regulator fluidly coupled to the electronic pressure regulator, the electronic pressure regulator configured to regulate a pressure of a stream of air provided by the air source; and
a pressure sensor coupled to a fluid line downstream of the electronic pressure regulator, wherein the electronic pressure regulator is controlled based on one or more signals generated by the pressure sensor,
wherein the air supply system is configured to provide pressurized air to the pulse generator of the pulse generation system based on one or more signals generated by at least one of the pressure sensor or the flow sensor.

15. The system of claim 1, wherein the medical condition comprises at least one of a hemorrhage, a heart attack, anemia, an ischemic stroke, peripheral vascular disease, trauma, or respiratory failure.

16. A method of performing perfusion of a circulatory system of a mammal, the method comprising:
inserting a cannula of a perfusion system into an arterial blood vessel of a mammal experiencing at least one medical condition;
controlling a pulse generation system to provide pulsatile flow of perfusate along a fluid line fluidly coupled to the cannula; and
adjusting the pulsatile flow of the perfusate based on signals generated by at least one of a pressure sensor coupled to the fluid line or a flow sensor coupled to the fluid line.

17. The method of claim 16, wherein the signals generated by at least one of a pressure sensor coupled to the fluid line or a flow sensor coupled to the fluid line indicate an internal resistance of an arterial system of the mammal.

18. The method of claim 16, wherein:
the pulse generation system comprises a pulse generator and an air supply system fluidly coupled to the pulse generator; and
adjusting the pulsatile flow of the perfusate comprises controlling the air supply system to supply pressurized air to pulse generator at a particular frequency determined based on signals generated by the at least one of the pressure sensor or the flow sensor.

19. The method of claim 16, further comprising controlling at least one of (i) a temperature of the perfusate to using a heat exchanger of the perfusion system, (ii) a concentration of oxygen in the perfusate using a gas mixer of the perfusion system, or (iii) controlling a syringe pump manifold to add one or more therapeutic compounds to the perfusate.

20. The method of claim 19, wherein:
the method further comprises monitoring one or more vital signs of the mammal; and
controlling the syringe pump manifold to add the one or more therapeutic compounds to the perfusate comprises controlling the syringe pump manifold based on the one or more vital signs.

21. The method of claim 19, wherein the one or more therapeutic compounds comprise an enhanced oxygen carrier compound.

22. The method of claim 21, wherein:
the method further comprises measuring an oxygen saturation of the mammal using at least one of (i) a vital sign sensor coupled to the mammal or (ii) a hematocrit detector coupled to the fluid line; and
the syringe pump manifold is controlled based on the measured oxygen saturation of the mammal.

23. The method of claim 16, wherein adjusting the pulsatile flow of the perfusate based on signals generated by at least one of a pressure sensor coupled to the fluid line or a flow sensor coupled to the fluid line comprises:
monitoring a blood pressure of the mammal based on data generated by the pressure sensor; and
adjusting the pulsatile flow of the perfusate based on the blood pressure of the mammal.

24. The method of claim 16, wherein:
the method further comprises monitoring one or more vital signs of the mammal using at least one vital sign sensor; and
the pulsatile flow of the perfusate is adjusted further based on data generated by the at least one vital sign sensor.

25. The method of claim 16, wherein adjusting the pulsatile flow of the perfusate based on signals generated by at least one of a pressure sensor coupled to the fluid line or a flow sensor coupled to the fluid line comprises providing the signals generated by the at least one of the pressure sensor or the flow sensor to a trained machine learning model.

26. The method of claim 25, further comprising training the machine learning model, wherein training the machine learning model comprises:
inputting a plurality of data generated by at least one vital sign sensor, the pressure sensor, and the flow sensor to the machine learning model as training data; and
training the machine learning model, based on the training data, to detect one or more vital signs of the mammal based on data generated by the at least one of the pressure sensor or the flow sensor.

27. The method of claim 16, wherein the mammal is a human or a pig.

28. The method of claim 16, wherein the medical condition comprises at least one of a hemorrhage, a heart attack, anemia, an ischemic stroke, peripheral vascular disease, trauma, or respiratory failure.
